(19) United States
(12) Patent Application Publication
(10) Pub. No.: US 2007/0260224 A1
(43) Pub. Date: Nov. 8, 2007

(54) FLEXIBLE CATHETER TIP HAVING A SHAPED HEAD

(75) Inventors: Randolf Von Oepen, Los Altos, CA (US); Thomas Michael Rieth, Hirrlingen (DE); Lorcan James Coffey, Tubingen (DE); Richard Roy Newhauser, San Francisco, CA (US); Travis Richard Yribarren, San Mateo, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: Abbott Laboratories, Santa Clara, CA (US)

(21) Appl. No.: 11/683,997

(22) Filed: Mar. 8, 2007

Related U.S. Application Data
(60) Provisional application No. 60/780,752, filed on Mar. 9, 2006.

(51) Int. Cl. A61M 25/16

(52) U.S. Cl. 604/528

(57) ABSTRACT

A catheter tip for attachment with an intravascular balloon catheter that is used within a body lumen is disclosed. The catheter tip includes a body portion having a base that is attached to the balloon catheter. The base defines a proximal end of the catheter tip. A head that is attached to the body portion defines a distal end of the catheter tip. An axial cavity, cooperatively defined by the body portion and the head, extends longitudinally from the proximal to the distal end of the catheter tip. The axial cavity receives a first guidewire along which the catheter is moved within the body lumen. An outer surface of the head is shaped in a predetermined manner to control the magnitude of deflection of a second guidewire that is often twisted about the first guidewire in the lumen so as to prevent catheter obstruction by the guidewires during catheter procedures.

### CROSS-REFERENCE TO RELATED APPLICATIONS

This application claims the benefit of the U.S. Provisional Patent Application No. 60/780,752, filed Mar. 9, 2006, and entitled “Contoured Stent and Delivery System with Novel Tip Design,” which is incorporated herein by reference in its entirety.

### BACKGROUND

1. Technology Field

The present invention generally relates to intraluminal catheters, such as balloon and stent delivery catheters, used in intravascular systems. In particular, the present invention relates to a catheter tip having features that facilitate intraluminal passage of the catheter when twisted guidewires are present in the passage.

2. The Related Technology

Angioplasty and stent implantation procedures are commonly employed to treat lesions or blockages that form within the vascular anatomy of a patient. During an angioplasty, or percutaneous transluminal coronary angioplasty (“PTCA”) procedure, for instance, a guiding catheter is advanced through the vasculature of the patient to a desired point, such as the ostium of a predetermined coronary artery. A guidewire, positioned within a balloon catheter, is extended from a distal end of the guiding catheter into the patient's coronary artery until it penetrates and crosses a lesion to be dilated. The balloon catheter is then advanced through the guiding catheter and over the previously introduced guidewire, until it is properly positioned across the lesion.

Once properly positioned, the balloon is inflated to a predetermined size such that the stenosis of the lesion is compressed against the arterial wall, thereby expanding the passageway of the artery. The balloon is subsequently deflated, blood flow resumes through the dilated artery, and the balloon catheter is removed.

Occasionally, post-procedure restenosis, or reformation of the arterial blockage, occurs after the PTCA procedure has been performed. To reduce the incidence of restenosis and strengthen the dilated area, physicians frequently implant an intravascular prosthesis, generally called a stent, inside the artery at the site of the lesion. During a stent implantation procedure, a stent is delivered in a contracted state on a balloon catheter to the desired location within a coronary artery. Once properly positioned, the stent is expanded to a larger diameter via expansion of the balloon, which causes the stent to expand against the arterial wall at the lesion site. The balloon is then deflated and it and the catheter are withdrawn. The expanded stent remains in place within the artery at the site of the dilated lesion, holding the vessel open and improving the flow of blood therethrough.

Lesions are often located at or near a point of bifurcation in an artery or other body vessel. When treating such bifurcated lesions, it is common to first place a first guidewire in the main branch, then place a second guidewire, extending from the main branch, into the side branch of the vessel bifurcation. During its placement, it is often necessary to rotate, or torque, the second guidewire in order for the tip thereof to engage the ostium of the side branch. Torquing the second guidewire in this manner occasionally results in the second guidewire intertwining with the first guidewire such that twists between the two guidewires are present within the main vessel branch proximate the bifurcated lesion region.

Intertwining of the two guidewires within the vessel as described above can significantly complicate proper placement of the balloon catheter at the bifurcated lesion region. Specifically, when it is tracked over the first guidewire, the balloon catheter meets with these twists of the second guidewire about the first guidewire. If the balloon catheter is then advanced further along the first guidewire, a “plowing” effect occurs, wherein the twisted second guidewire is slid along with the balloon catheter as it proceeds distally along the first guidewire. Undesirably, this causes a tightening of the twists of the second guidewire about the first guidewire within the vessel.

Should the second guidewire twists become too tight about the first guidewire, a pinching force can be imposed by the second guidewire on the tip of the balloon catheter, thereby impeding or preventing further advancement of the balloon catheter within the vessel. The unfortunate results of this situation include compromised catheter placement, possible damage to vessel structure, etc.

In light of the above discussion, a need exists in the art for a catheter system capable of use with multiple guidewires employed in treating intravascular lesions at bifurcated regions. In particular, a catheter configuration is needed that alleviates problems occasioned by the advancement of the catheter along a first guidewire when a second guidewire is twisted thereabout. Any solution to the above need should prevent or reduce plowing of the twisted second guidewire as the catheter is advanced, so as to avoid binding of the catheter tip caused by the tightening of the second guidewire twists. Moreover, any proposed solution should be adaptable for use with a variety of catheter types and configurations.

### BRIEF SUMMARY

The present invention has been developed in response to the above and other needs in the art. Briefly summarized, embodiments of the present invention are directed to a catheter tip for attachment with an intravascular balloon catheter that is used within a body lumen. The catheter tip includes a body portion having a base that is attached to the balloon catheter. The base defines a proximal end of the catheter tip. A head that is attached to the body portion defines a distal end of the catheter tip. An axial cavity, cooperatively defined by the body portion and the head, extends longitudinally from the proximal to the distal end of the catheter tip. The axial cavity receives a first guidewire along which the catheter is moved within the body lumen. An outer surface of the head is shaped in a predetermined manner to control the magnitude of deflection of a second guidewire that is often twisted about the first guidewire in the lumen so as to prevent catheter obstruction by the guidewires during catheter procedures. The outer surface of the catheter tip head can define various shapes, including rounded, tapered, and bulbous shapes.

The body portion is defined by a coiled wire having a predetermined pitch. This coiled configuration lends flexibility to the catheter tip, enabling it to ease passage of the balloon catheter through the body lumen. In one embodiment, the body portion of the tip is z encapsulated with a covering material that further optimizes passage of the balloon catheter.

These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof that are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A is a side view of a catheter tip configured in accordance with one embodiment of the present invention;

FIG. 1B is an end view of a proximal end of the catheter tip of FIG. 1A;

FIG. 1C is an end view of a distal end of the catheter tip of FIG. 1A;

FIG. 2 is a perspective view of a catheter tip having a head configured according to one embodiment;

FIG. 3 is a perspective view of a catheter tip having a head configured according to another embodiment;

FIG. 4 is a simplified side view of a catheter having a catheter tip according to one embodiment, wherein the catheter is tracking along a first guidewire that is twisted with a second guidewire within a vessel lumen;

FIG. 5 is a close-up view of a catheter tip configured in accordance with one embodiment tracking along a first guidewire, and further illustrating the manner in which the catheter tip prevents tightening of a twisted second guidewire;

FIG. 6 is a perspective view of a catheter tip configured according to yet another embodiment;

FIG. 7 is a cross sectional side view of tapered tube stock from which the catheter tip of FIG. 6 can be produced;

FIG. 8 is a cross sectional side view of a catheter tip encased in a covering material and attached to a catheter, according to another embodiment;

FIGS. 9A-9C are side views of catheter tips having body portions that are configured according to yet other exemplary embodiments; and

FIG. 10 is a cross sectional side view of an encapsulated catheter tip configured according to yet another embodiment.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the invention, and are not limiting of the present invention nor are they necessarily drawn to scale.

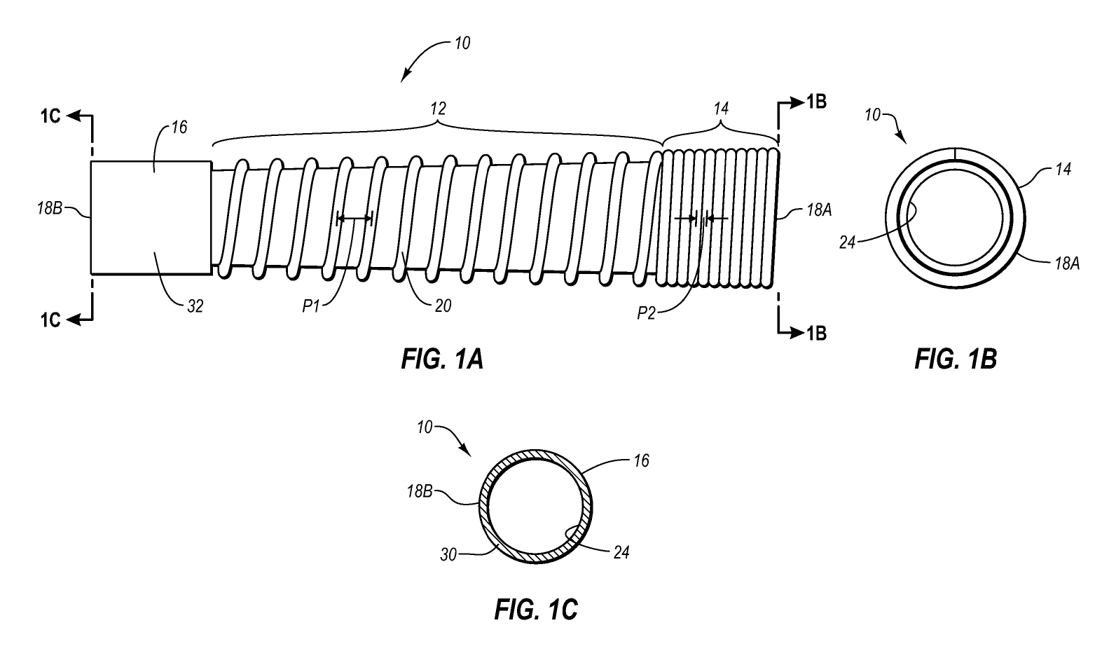

FIGS. 1-10 depict various features of embodiments of the present invention, which is generally directed to a tip for use with intravascular catheters employed in angioplasty and stent implantation procedures. The catheter tip is attached to a distal end of the catheter and receives a first guidewire already positioned in the lumen of the body vessel, such as a coronary artery, to guide the tracking of the catheter along the first guidewire.

The catheter tip is further configured to enable the catheter to track through vessels where a second guidewire is positioned with the first guidewire in a main branch and extends into a side branch of the vessel, known as a bifurcated lumen, to treat a lesion proximate the bifurcation. As the second guidewire often twists about the first guidewire in the main vessel branch, the catheter tip is shaped and configured to enable passage of the catheter through such regions without causing a tightening of the second guidewire twisting, which can cause wedging of the catheter between the guidewires and prevent advancement of the catheter to the lesion. Further, the catheter tip is designed so as to prevent pinching of the first guidewire by the tip, another common consequence of twisting between the first and second guidewires. In addition, embodiments of the present invention provide for a flexible tip design, thereby easing advancement of the catheter through the vessel lumen.

Reference is first made to FIGS. 1A-1C, which shows various details of a tip, generally designated at 10, configured in accordance with one embodiment for placement at a distal end of a catheter (not shown here). As shown, the catheter tip 10 generally includes a body portion 12, a base portion 14, and a head 16. The base portion 14 defines a proximal end 18A of the catheter tip 10, while the head 16 defines a distal end 18B of the tip. So configured, the proximal end 18A is adapted for attachment with a catheter, while the distal end 18B serves as the most distal portion of the catheter assembly when the tip 10 is mated with the catheter, and as such is lead portion of the catheter when tracking through the lumen of a vessel, such as a coronary artery.

As best shown in FIG. 1A, the body and base portions 12 and 14 of the tip 10 are defined by an elongate coiled wire 20 that extends from the proximal end 18A to the base of the head 16. In the illustrated embodiment, the coiled wire 20 has a diameter of approximately 0.05 mm and a pitch that varies according to the portion of the tip 10 it defines. The portion of the coiled wire 20 that defined the base portion has a pitch P1 of approximately 0.05 mm, while the pitch of the coiled wire portion defining the body portion 12 has a pitch P2 of approximately 0.15 mm. Of course, the diameter and respective pitches of the coil can be modified from what is described herein, according to need for a particular application. For example, and not by way of limitation to the above, the diameter of the coil wire can range from about 0.01 mm to about 1 mm, while the pitch can range from about 0.01 mm to about 10 mm, depending upon the particular application, including coronary artery, non-coronary artery, and other body vessel applications. This results in a coiled wire body portion having a maximum diameter range of about 0.62 mm to about 0.66 mm in one embodiment.

The wire from which the coil 20 is formed is stainless steel in the present embodiment. In other embodiments, other suitable materials can be used to compose the wire. For instance, and not by way of limitation to the possible materials usable to form coil 20, the coil 20 can be formed from a metal, metal alloys, shape memory material, polymer, plastic, synthetic material, natural material, combinations thereof, or other material having the desired material characteristics and properties to aid with moving the catheter along the guidewire. Also, though the cross sectional shape of the wire here is circular, other cross sectional shapes can alternatively be used.

The coil 20 is further shaped such that both the body and base portions 12 and 14 of the tip 10 define a decreasing taper from the proximal end 18A to the point of attachment with the head 16. In the present embodiment the taper has a magnitude of approximately 1.5 degrees, though other taper amounts can alternatively be used. For example, and not by way of limitation to the above, the taper can have an angular orientation of from about 0 degrees to about 30 degrees, depending upon the particular application.

The tapered coil described above lends flexibility to the catheter tip 10, which in turn assists the catheter in tracking along the relatively tortuous vasculature of a patient's body during the angioplasty, stent placement, or other procedure. The taper defined by the coiled wire 20 of the body and base portions 12 and 14 further shapes the tip for relative ease in catheter advancement.

It is noted that, though separately identified above, the base portion can be integrally formed with the body portion of the catheter tip in the present embodiment. In other embodiments, however, the base portion, like the head in the above embodiment, can be separately defined then attached to the body portion of the tip. Moreover, the coiled wire shown in FIG. 1A is continuously defined from a single wire strand, but other wire configurations, such as joined or overlapping wires, can be used to define the body and/or base portions of the tip.

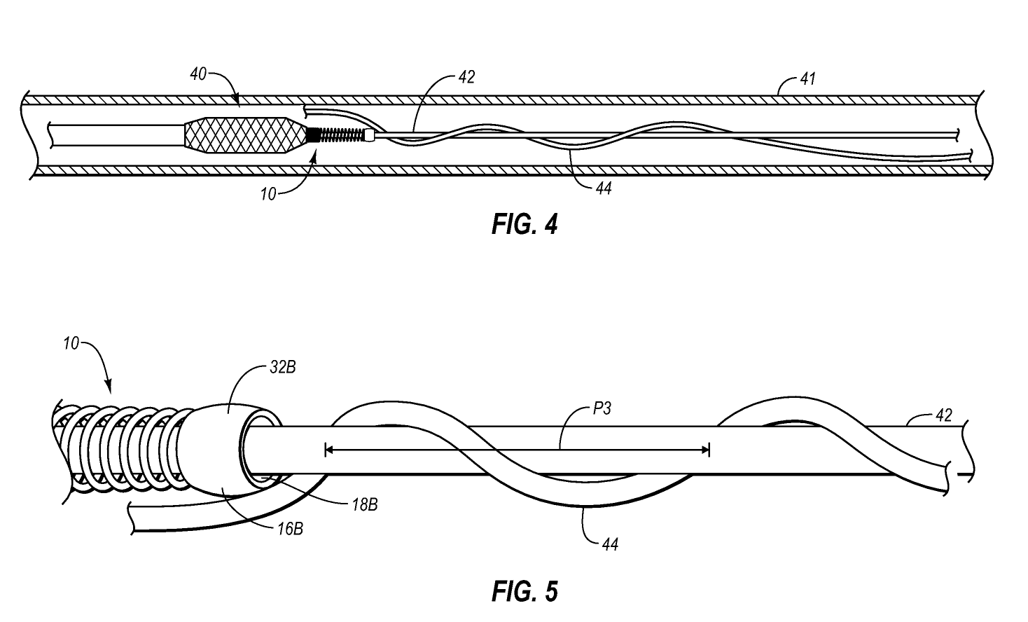

The body portion 12, base portion 14, and head 16 cooperate to define an axial cavity 24 that longitudinally extends between the proximal and distal ends 18A-B of the tip 10. The cavity 24 is configured to receive a first guidewire (FIG. 4) during intravascular travel, enabling direction of the catheter to the lesion site.

The distal end 18B of the head 16 includes an annular face 30 circumscribing the opening of the cavity 24. As illustrated in FIGS. 1A and 1C, the face 30 is flat, though in other embodiments it can have a beveled, rounded, or other contoured shape, as will be seen, to assist intravascular catheter travel.

The head 16 further defines an outer surface 32. In accordance with the present embodiment, the head 16 has a hollow cylindrical shape with the outer surface 32 defining an outer head diameter of approximately 0.50 mm, and the axial cavity 24 of this portion being defined by an inner head diameter of approximately 0.42 mm. The axial length of the head outer surface 32 here is approximately 0.50 mm. Though defining a cylindrical shape here, the head outer surface in other embodiments can be shaped so as further assist catheter travel through the vessel, as will be seen further below. Further, these dimensions, of course, are exemplary, and should not be construed as limiting the present invention in any way. For example, and not by way of limitation, the outer head diameter can range from about 0.4 mm to about 10 mm, the inner head diameter can range from about 0.2 mm to about 9 mm, and the axial length of the head out surface can range from about 0.05 mm to about 50 mm.

Like the coiled wire 20, the head 16 can be formed from stainless steel or other suitable material. For instance, and not by way of limitation to the possible materials usable to form head 16, the head 16 can be formed from a metal, metal alloys, shape memory material, polymer, plastic, synthetic material, natural material, combinations thereof, or other material having the desired material characteristics and properties to aid with moving the catheter along the guidewire. A suitable material includes one whose hardness prevents deformation of the head during intravascular travel and interaction of the tip with the guidewires disposed within the vessel. This in turn enables the size of the cavity 24 defined through the tip 10 to be reduced so as to minimize the clearance between the outer diameter of the guidewire and the inner diameter of the head 16, as additional cavity clearance to compensate for deformation of the head—which otherwise would cause binding of the head on the guidewire—is not necessary.

Manufacture of the head from a relatively hard material also enables the radial thickness of the head 16 to be minimized as well. This in turn reduces the annular thickness of the annular face 30 of the head 16, which assists the tip 10 in passing through guidewire twists or penetrating obstructing lesions within the vessel, as will be explained.

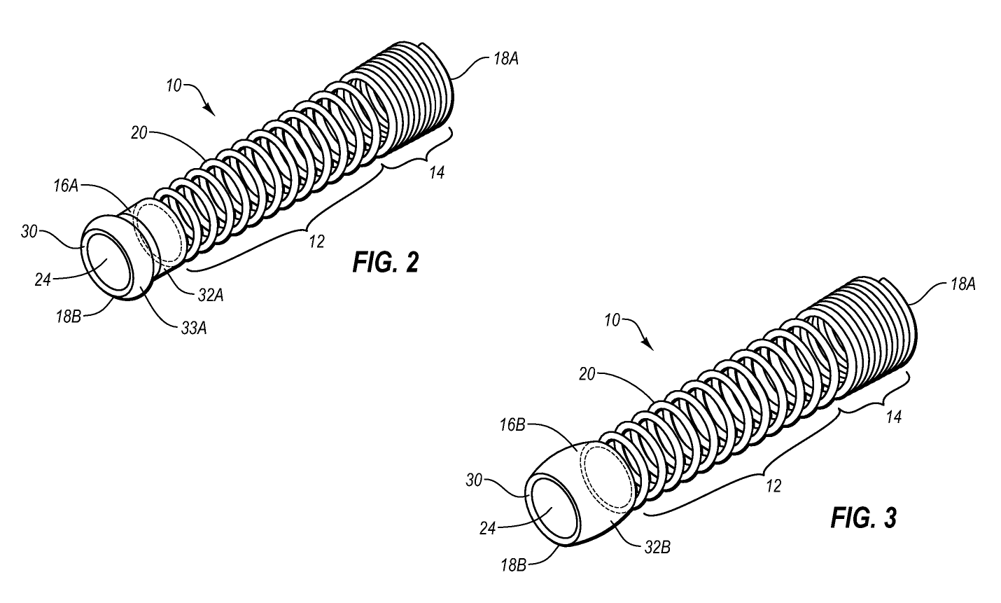

Reference is now made to FIGS. 2 and 3, which depicts details regarding the catheter tip 10 having head designs according to other embodiments of the present invention. In particular, FIG. 2 shows a catheter tip 10 having a head 16A attached to the body portion 12 as in the embodiment shown in FIGS. 1A-1C. In contrast to that embodiment, however, the head 16A includes an outer surface 32A having a bulbous extended portion 33 proximate the distal end 18B. Similarly, the catheter tip 10 shown in FIG. 3 includes a head 16B having an outer surface 32B. The entirety of the outer surface 32B defines a rounded olive-shaped, bulbous surface. Also, and as was the case with the head 16 in FIGS. 1A-1C, the heads 16A and 16B can be composed of stainless steel or another suitable hard material so as to prevent deformation thereof during intravascular use. For instance, and not by way of limitation to the possible materials usable to form head 16, the heads 16A and 16B can be formed from a metal, metal alloys, shape memory material, polymer, plastic, synthetic material, natural material, combinations thereof, or other material having the desired material characteristics and properties to aid with moving the catheter along the guidewire. The tip head hardness, together with the shaped outer head surface as described above, assists in catheter advancement through the vessel lumen, as further discussed in connection with FIGS. 4 and 5, below.

Reference is now made to FIGS. 4 and 5 in describing operation of a catheter equipped with a catheter tip configured in accordance with an embodiment of the present invention. Indeed, FIG. 4 shows a balloon catheter 40 disposed within a main branch of a lumen of a body vessel, such as an artery 41. A first guidewire 42 is also disposed in the lumen, having been positioned prior to catheter insertion into the vessel. The catheter 40 includes a catheter tip 10 configured in accordance with the embodiment as shown in FIG. 3, for example. The first guidewire 42 is received through the catheter and the cavity 24 of the catheter tip 10 so as to enable the catheter to track along the first guidewire as it advances through the vessel lumen to the lesion site.

A second guidewire 44 is also shown disposed within the vessel lumen. The first guidewire 42 is positioned in a main branch of the vessel, while the second guidewire—though having a portion in the main branch as shown in FIG. 4—extends into the lumen of a vessel side branch (not shown) that forms a bifurcation with the main branch of the vessel. Placement of the second guidewire in the side branch of the bifurcated vessel is often desired or necessary in order to treat lesions located proximate the vessel bifurcation.

To position the guidewires as described above, the first guidewire 42 is first inserted into the main vessel branch to a predetermined location. The second guidewire 44 is then introduced into the main vessel branch and advanced until it is positioned within the side branch of the vessel at the point of bifurcation. In order to maneuver the second guidewire to entry into the side branch, it is often necessary to rotate, or torque, the second guidewire as it advances through the main vessel branch. Such torquing of the second guidewire 44 typically results in the second guidewire loosely twisting about the first guidewire 42 in the main vessel branch. This situation is shown in FIG. 4.

Once the first and second guidewires 42 and 44 are positioned in the main and side vessel branches, respectively, the catheter 40 is introduced into the vessel and tracks along the first guidewire as it is advanced through the main branch toward the point of vessel bifurcation. As it advances along the first guidewire 42, the catheter 40 encounters the twists of the second guidewire 44 about the first guidewire.

The tip 10 of the catheter 40 is configured to enable the catheter to advance through and past such guidewire twists without causing a tightening of the twists, which would otherwise progress of the catheter. Specifically, and as shown in FIG. 5, the bulbous shaped outer surface 32B of the tip head 16 is configured such that when a twist of the second guidewire 44 is incident on the tip head—which head is generally the point of first contact of the second guidewire with the catheter—the twist is redirected in such a way as to enable the catheter to slide past and through the twist. Significantly, the tip 10 and rest of the catheter 40 slides past the twist in the second guidewire 44 without causing a tightening of the twist and succeeding twists in the second guidewire. This is made possible by the contoured shape of the outer surface 32B of the tip head 16B, which minimizes redirection of the second guidewire upon contact with the tip head. As such, the pitch of the second guidewire twists, approximately indicated at P3 in FIG. 5, is not significantly reduced, which reduction would indicate a tightening of the second guidewire twists about the first guidewire 42. This result should be compared with known catheter tips, which include more abrupt tip profiles and which undesirably cause abrupt redirection of the second guidewire, resulting in reduction of twist pitch, i.e., tightening of the twists thereof about the first guidewire. As has been mentioned, such tightening of the twists can result in wedging of the catheter between the first and second guidewires, impeding progress of the catheter.

Advancement of a catheter having a catheter tip as described in accordance with embodiments of the present invention is further assisted by the head 16B being composed from a relatively hard material, such as stainless steel. In instances where some slight wedging of the catheter 40 between the first and second guidewires 42 and 44 occurs, the relative hardness of the tip head 16B ensures that deformation thereof will not result, which as mentioned could otherwise cause the head to bind on the first guidewire passing therethrough.

Further, the relative hardness of the tip head 16B enables it to be made with a minimized clearance for the axial cavity 24, thereby further reducing the overall profile of the tip head 16, which in turn increases the ability of the catheter 40 to pass through the second guidewire twists or to penetrate an obstructing vessel lesion. Moreover, the general flexibility of the catheter tip body and base portions 12 and 14 provided by the coiled wire 20 further assists catheter passage through the vessel lumen.

The passage process of the catheter 40 past the second guidewire twists continues during catheter advancement through the vessel until all of the twists have been passed or until the catheter is properly positioned at a lesion site. Note that, though the catheter tip head design shown in FIGS. 4 and 5 is that represented in FIG. 3 of the accompanying drawings, this is exemplary only, and the other catheter tip designs shown and described herein can also be used to gain the benefits discussed above.

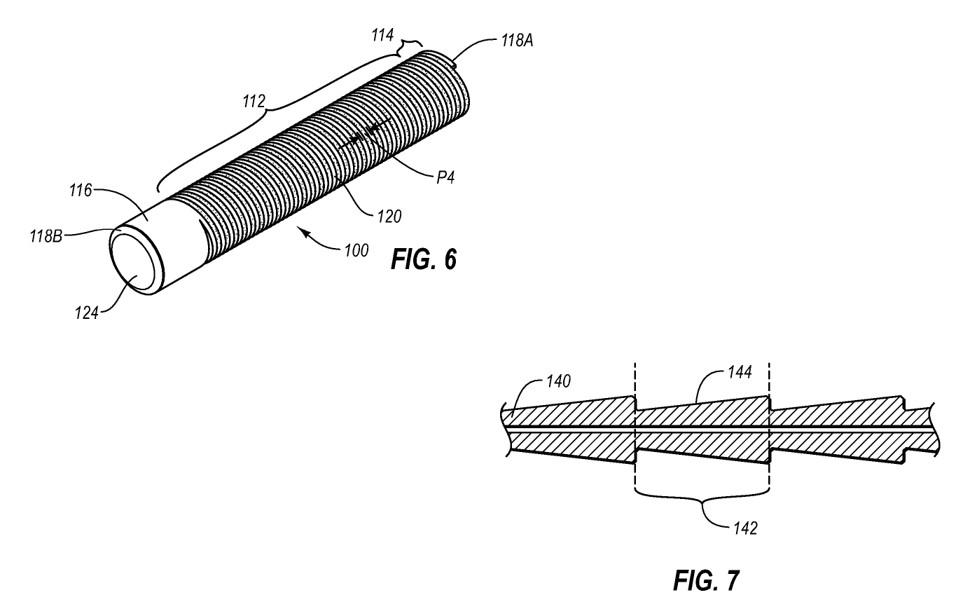

Reference is now made to FIG. 6 in describing a catheter tip configured according to one embodiment. In detail, a catheter tip 100 is disclosed. The tip 100 includes a body portion 112 with an integrally formed base portion 114 and an integrally formed head 116 defining a proximal end 118A and a distal end 118B, respectively. As in previous embodiments, the body and base portions 112 and 114 include a coiled configuration, though the method of coil definition in the present embodiment differs therefrom. Particularly, the tip 100 is formed from a hollow, tapered cylinder having a coil 120 defined therein. The coil 120 is defined by laser cutting through the cylinder in a spiral pattern from the proximate end 118A to the head 116.

In the present embodiment, the laser cut achieved is a cross sectionally square cut having approximate dimensions of 0.05 mm×0.05 mm. The laser cut is further made so as to impart a 0.25 mm pitch P4 to the coil 120. The coil 120 can be composed of three separate laser cut coils spirally defined adjacent one another, though in other embodiments fewer or more coils can be defined in the tip 100, including the possibility of a single continuous spiral cut to define the coil. Again, the tip 100 has a taper shape, tapering down from the proximal end 118A to the head 116. The taper, pitch, and other dimensions of the tip and laser cut of the coil can, of course, be modified from what is described herein. For example, and not by way of limitation, the cross-sectional square cut dimensions can range from about 0.01 mm×0.01 mm to about 1 mm×1 mm, while the pitch can range from about 0.01 mm to about 10 mm, depending upon the particular application. Further, it will be understood that the laser cut need not have a square cut, but can have various other cut configurations, such as rectangular for instance.

As the tip 100 is manufactured from a single stock piece, the head 116 is integrally formed with the body portion 112 and base portion 114. These components therefore integrally define an axial cavity 124 extending longitudinally between the proximal and distal ends 118A and 118B.

FIG. 7 shows a portion of tube stock 140 from which the tip 100 shown in FIG. 6 may be manufactured, in one embodiment. The tube stock 140 includes various segments 142, each segment having been shaped with a taper 144—via grinding, laser cutting, or other suitable method—to match the taper of the tip 100 when complete. The laser cut coil 120 (FIG. 6) can be defined on the segment 142 either before or after separation from the other segments of the tube stock 140. Note that this figure illustrates only one possible configuration for producing a catheter tip; other viable configurations for producing the tips described herein exist. The design of the catheter tip 100 shown in FIG. 6 desirably improves trackability of the catheter while disposed intravascularly as it can be modified in design to exhibit specific stiffness characteristics.

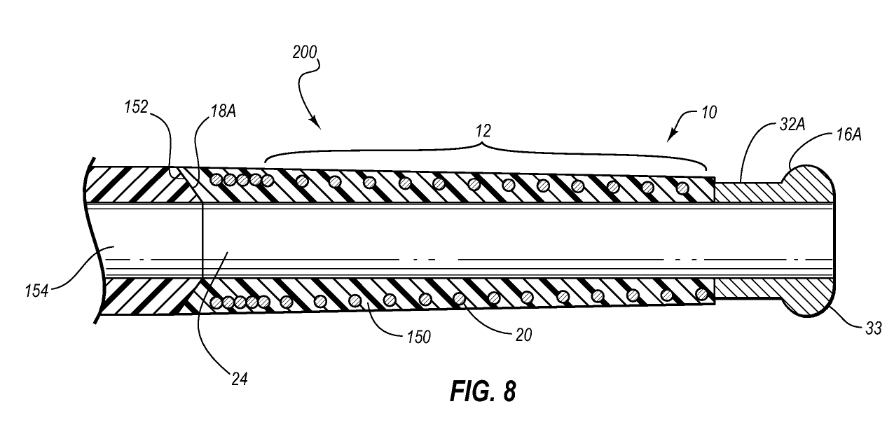

Reference is now made to FIG. 8, wherein an encapsulated catheter tip assembly, generally designated at 200, is shown. The encapsulated tip assembly 200 includes the catheter tip 10 similar to that shown in FIG. 2, including the body portion 12 defined by the coiled wire 20 and head 16A that defines the contoured outer surface 32A. The encapsulated tip assembly 200 is useful in providing a relatively smoother body and base outer surface for easing tracking and catheter advancement along the first guidewire.

The body and base portions 12 and 14 of the catheter tip 10 are substantially z covered by a covering material, or encapsulant 150, composed of a suitable material such as plastics and polymers, including but not limited to PEBAX, vinyl, nylon, polyurethane or other materials suitable for use with the catheter. The head 16A can remain uncovered. The encapsulant 150 can be applied to the catheter tip 10 so as to preserve the desired dimensions of the cavity 24 and outer surface portions of the base and body portions 12 and 14. Application of the encapsulant 150 to the catheter tip 10 can occur in one of several ways, including molding, dipping, etc., and can occur either before or after the tip is attached to the catheter.

Though its use has been discussed herein in connection with its use in PTCA and stent implantation procedures, the catheter tip and associated catheter can also be modified for use in treating chronic total occlusions (“CTOs”), wherein the relatively hard head of the tip enables penetration through the fibrous cap commonly present with such occlusions—something not always possible with softer tipped catheters.

Note that a catheter tip configured in accordance with the embodiments described herein can be attached to the balloon or other suitable catheter in one or more of a variety of ways including, for example, friction fit, thermal or chemical adhesive, mechanical fasteners, circumferential connectors, etc. In the latter case, for instance, a band of wire or other suitable connector can be secured about the base portion 14 of the catheter tip 10 shown in FIG. 1A, such that the catheter base portion is secured in a friction, or interference, fit with an end of the balloon catheter. To accommodate such a fastening, the catheter tip base portion can be modified in shape—such as providing an annular concavity about the outer surface of the base portion—as needed to provide a suitable fastening surface.

FIG. 8 further shows one manner of attachment between a catheter tip, such as the tip 10 shown here, and a distal portion of a catheter, such as the catheter 40 shown in FIG. 4. In particular, the catheter 40 includes in one embodiment a distal portion 152 composed of a polymeric material. The catheter distal portion 152 and the encapsulant 150 defining the proximal end 18A of the catheter tip 10 can be heated so as to enable the corresponding surfaces thereof to bond with one another. These corresponding surfaces can be further shaped so as to correspondingly mate with one another, further enhancing their union. As one example of such shaping, the catheter distal portion 152 and the proximal end 18A of the catheter tip in FIG. 8 define complementary annular frustoconical surfaces. Of course, other complementary mating surfaces can also be used. Note that the joining of the catheter tip to the catheter is such that an axial cavity 154 of the catheter 40 is aligned with the catheter tip axial cavity 24.

In those embodiments where the catheter tip is not encapsulated, the distal portion of the catheter can be softened by heating and the proximal end of the catheter tip inserted therein such that a portion of the proximal end is embedded in the distal portion of the catheter, thereby joining the two components.

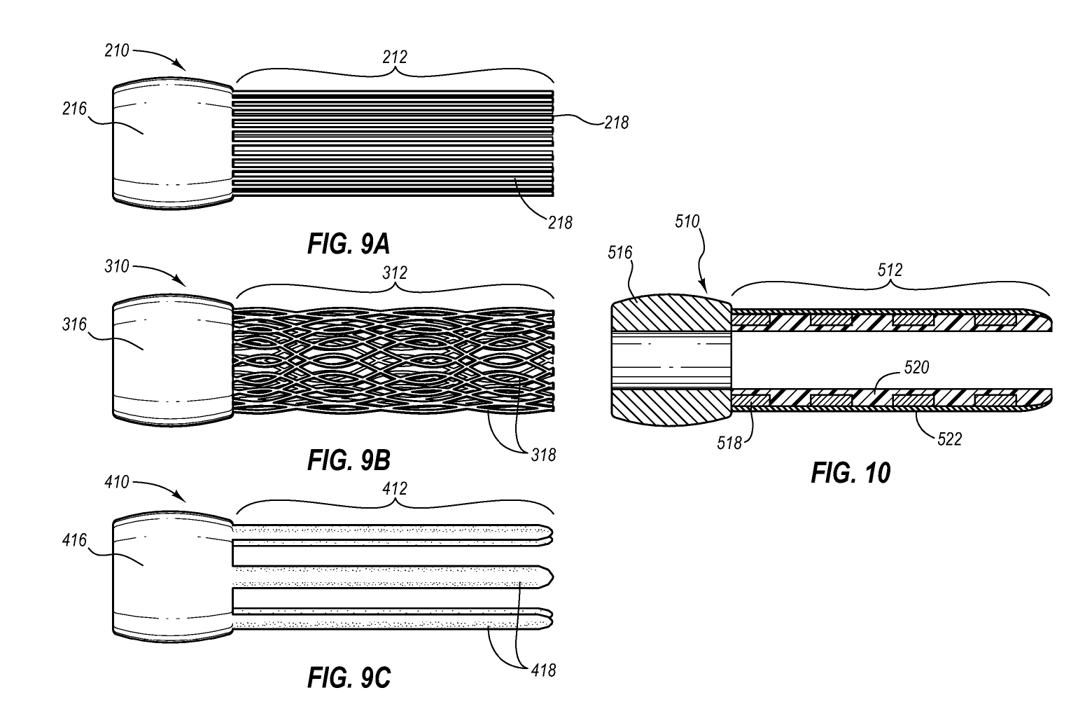

Reference is now made to FIGS. 9A-9C, which depict various additional configurations for the body portion of the catheter tip, according to exemplary embodiments of the present invention. Specifically, FIG. 9A shows a catheter tip 210 having a body portion 212 and head 216. The body portion 212 is composed of a plurality of elongate fingers 218 extending from the head 216. In one embodiment, the fingers are integrally formed with the head 216 and are defined by a laser cutting or other suitable process. The fingers 218 can remain in the configuration shown in FIG. 9A, or can be twisted into a spiral configuration to add additional flexibility to the tip.

In FIG. 9B, a catheter tip 310 is shown, having a body portion 312 and head 316. The body portion 312 is defined by a plurality of elongate fingers 318 attached by welding or other joining method and braided into a cylindrical lattice configuration, offering flexibility to the body portion 312.

Yet another catheter tip configuration is shown in FIG. 9C, which depicts a catheter tip 410 having a body portion 412 and head 416. The body portion 412 is defined by a plurality of two or more fingers 418 having a relatively thicker configuration than that shown in FIG. 9A or 9B. The fingers 418 can be sandblasted or otherwise given surface featuring to facilitate their encapsulation by an encapsulant, such as that shown at 150 in FIG. 8. Alternatively, the fingers 418 can be configured to slip under a sleeve defined at the distal end of a catheter, such as the balloon catheter shown at 40 in FIG. 4. The number of fingers 418 included can be between two and five, or more in other embodiments.

Though the embodiments shown herein include catheter tips composed of a metal, in one embodiment the catheter tip head and body portion can be formed from a polymeric material, such as PEEK, for instance. Such a composition could be especially applied to the braided catheter tip configuration shown in FIG. 9B.

Reference is now made to FIG. 10, which shows yet another manner of attachment between a catheter tip and a distal portion of the catheter, such as the balloon catheter 40 shown in FIG. 4. Particularly, a catheter tip 510 is shown, having a body portion 512 and head 516. The body portion 512 includes a square coiled wire 518. To join the catheter tip to the catheter, the body portion 512 is slipped over a polymeric distal portion 520 of the catheter. The distal portion 520 is then heated, thereby partially encapsulating the coiled wire 518 and joining the two components. If desired, an outer encapsulant sleeve 522 composed of polymer of other suitable substance is positioned over an exterior surface of the body portion 512 and heated to bond the outer encapsulant sleeve with the coiled wire 518, thereby embedding the coiled wire between the polymeric outer encapsulant sleeve and the distal portion 520 in a sandwich configuration. This ensures the body portion 512 of the catheter tip 510 is fully covered, yet flexible.

The present invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. A catheter tip attached to an intravascular balloon catheter used within a body lumen, the catheter tip comprising:
a body portion having a base that is attached to the balloon catheter, the base defining a proximal end of the catheter tip;
a head attached to the body portion, the head defining a distal end of the catheter tip;
an axial cavity cooperatively defined by the body portion and the head, the axial cavity extending longitudinally from the proximal to the distal end of the catheter tip, the axial cavity receiving a first guidewire along which the catheter is moved within the body lumen; and
an outer surface defined by the head, the outer surface of the head being shaped in a predetermined manner to control the magnitude of deflection of a second guidewire when the head outer surface is incident with the second guidewire, the second guidewire disposed in the body lumen and not received by the axial cavity, the second guidewire being twisted about the first guidewire.

2. The catheter tip as defined in claim 1, wherein at least a portion of the outer surface of the head defines a bulbous shape.

3. The catheter tip as defined in claim 1, wherein substantially the entirety of the outer surface of the head defines a bulbous shape.

4. The catheter tip as defined in claim 1, wherein at least a portion of the outer surface of the head is tapered.

5. The catheter tip as defined in claim 1, wherein the head further defines an annular face at the distal end, the annular face being adjacent the outer surface of the head, the annular face defining a beveled shape.

6. The catheter tip as defined in claim 1, wherein the body portion is flexible.

7. A balloon catheter for use within a body lumen, the balloon catheter comprising:
a catheter body; and
a catheter tip attached to the catheter body, the catheter tip including:
a head defining a distal end of the catheter tip;
a body portion having a base that defines a proximal end of the catheter tip, the body portion and head cooperating to define an axial cavity extending longitudinally between the proximal and distal ends of the catheter tip, wherein the body portion is defined by a material having a coiled configuration.

8. The catheter as defined in claim 7, wherein the material having the coiled configuration defining the body portion is flexible.

9. The catheter as defined in claim 7, wherein the material having the coiled configuration of the body portion defines a portion of the axial cavity.

10. The catheter as defined in claim 7, wherein the coiled configuration of the body portion is tapered.

11. The catheter as defined in claim 7, wherein the axial cavity receives a first guidewire for tracking the balloon catheter within the body lumen, and wherein the head includes a contoured outer surface configured to control deflection of a second guidewire twisted about the first guidewire and incident on the head.

12. The catheter as defined in claim 7, wherein the body portion is defined by a continuous elongate coiled wire.

13. The catheter as defined in claim 12, wherein the coiled wire is coiled such that the body portion has a first pitch and wherein the base has a second pitch.

14. The catheter as defined in claim 12, wherein the coiled wire is composed of stainless steel.

15. The catheter as defined in claim 7, wherein the body portion and head of the catheter tip are integrally formed and wherein the head has a tapered shape.

16. The catheter as defined in claim 15, wherein the coiled configuration of the body portion is at least partially produced by laser cutting the material defining the body portion.

17. A catheter tip for use with an intravascular balloon catheter used within a body lumen, the catheter tip comprising:
a body portion having a base that is attached to the balloon catheter, the base defining a proximal end of the catheter tip, the body portion having a flexible coil configuration;
a head attached to the body portion, the head defining a distal end of the catheter tip;
an axial cavity cooperatively defined by the body portion and the head, the axial cavity extending longitudinally from the proximal to the distal end of the catheter tip, the axial cavity receiving a first guidewire along which the catheter is moved within the body lumen; and
an outer surface defined by the head, the outer surface of the head being shaped in a predetermined manner to control the magnitude of deflection of a second guidewire when the outer surface is incident with the second guidewire, the second guidewire disposed in the body lumen and not received by the axial cavity, the second guidewire being twisted about the first guidewire.

18. The catheter tip as defined in claim 17, wherein the body portion is defined by a coiled wire composed of stainless steel, the body portion having a tapered configuration.

19. The catheter tip as defined in claim 18, wherein the coiled wire defines a first pitch at the base portion of the body portion and a second pitch at the body portion.

20. The catheter tip as defined in claim 19, wherein the head is composed of a material that is non-deformable during intravascular use of the balloon catheter.

21. The catheter tip as defined in claim 20, wherein the head is composed of stainless steel.

22. The catheter tip as defined in claim 21, wherein the outer surface of the head has a rounded shape.

23. The catheter tip as defined in claim 22, wherein the head further comprises a front face annularly defined about an opening of the axial cavity defined on the distal end of the tip, the front face being shaped to control the magnitude of deflection of the second guidewire.

24. The catheter tip as defined in claim 23, wherein the body portion is at least partially encapsulated by a covering material.

25. The catheter tip as defined in claim 24, wherein the covering material is composed of plastic.

26. An intravascular catheter tip, comprising:
a flexible body portion having a base configured for attachment with a distal portion of the catheter; and
a head attached to a body portion, the head being non-deformable and defining a distal end off the catheter tip.

27. The catheter tip as defined in claim 26, wherein the body portion includes a plurality of fingers integrally formed and with and extending from the head, the fingers being shapeable to define the body portion.

28. The catheter tip as defined in claim 26, wherein the body portion is defined by a plurality of fingers that are braided to form a cylindrical lattice.

29. The catheter tip as defined in claim 26, wherein the body portion includes a plurality of fingers that linearly extend from the head in an axial direction, the fingers having surface features thereon to facilitate an encapsulant being attached to at least a portion of the fingers.

30. The catheter tip as defined in claim 26, wherein the body portion is encapsulated by first and second encapsulating structures in a sandwich configuration.
